(19) United States
(12) Patent Application Publication
(10) Pub. No.: US 2008/0108642 A1
(43) Pub. Date: May 8, 2008

(54) Composition and its use as effective constituent of a medicine having anti-angiogenesis synergy effect

(75) Inventors: Shao Li, Beijing (CN); Min Wu, Beijing (CN)

Correspondence Address: GREER, BURNS & CRAIN, 300 S WACKER DR, 25TH FLOOR, CHICAGO, IL, 60606, US

(73) Assignee: Tsinghua University

(21) Appl. No.: 11/975,320

(22) Filed: Oct. 18, 2007

(30) Foreign Application Priority Data
Nov. 2, 2006 (CN) 200610114226X

(51) Int. Cl. A61K 31/4375; A61P 35/00

(52) U.S. Cl. 514/288

(57) ABSTRACT

The present invention relates to a composition as effective constituent of a medicine having anti-angiogenesis synergy effect and belongs to the field of medicine compositions. The recipe is composed of matrine and sinomenine hydrochloride with a content ratio of matrine:sinomenine hydrochloride=1:0.05 to 1. The recipe of the present invention has synergic actions of efficacy enhancing and toxicity reducing, has obvious therapy effect to angiogenesis-related diseases such as rheumatoid arthritis and cancer, has clear base of medicine effective substance, and maintain the characteristic of traditional Chinese medicine of recipe association.

### FIELD OF THE INVENTION

The present invention relates to the field of medicine compositions, particularly to a traditional Chinese herb-derived composition of active compounds as effective constituent having anti-angiogenesis synergy effect.

### BACKGROUND OF THE INVENTION

Angiogenesis is a common pathological process associated with various diseases such as rheumatoid arthritis (RA), cancer etc., which seriously endangers human health. An anti-angiogenesis medicine is an important way of treatment by blocking pathological changes of rheumatoid arthritis or “starving-to-death” tumor. For treatment of rheumatoid arthritis, for example, major medicines include hormones, gold salts, non-steroidal anti-inflammatory drugs (NSAIDS), and Chinese patent medicines. The first three kinds of medicines cannot block development of rheumatoid arthritis, and long-term administration of them will produce substantive side-effect. Chinese patent medicines have the defects of that their recipes are too big, that their effective constituents are not clear, and that their mechanisms are not clear. Tripterygium Hypoglaucum Hutch Tablet and Tripterygium Glycosides Tablet are two drugs with relatively clear constituent; they have immunodepression effect, but their serious damages to gastrointestinal tract, kidney and reproductive organs limit their applications.

QLY is a traditional Chinese medicine recipe for treatment of rheumatoid arthritis. It is mainly composed of four herbs, Sophora Root, Ovientvine, Amur Corktree Bark, and Hypoglaucous Collett Yan Rhizome (Rhizoma Dioscoreae Hypoglaucae). The recipe of QLY was first invented by a famous Chinese traditional doctor “ZHANG YI-TIE”, which has been handed down generation by generation among his descendants for over 300 years, and has been improved by his descendants during the over 300 years. Pharmacological experiments performed by one of the inventors of the present application, Shao LI, who is a descendant of “ZHANG YI-TIE”, found that QLY had the effect of anti-inflammatory, reducing synovial capillary vessel angiogenesis, preventing formation of pannus, alleviating cartilage damage, and reducing secretory function and metabolic activity of synovial cell hyperfunction; it also showed remarkable effect in suppressing angiogenesis in terms of molecular, organism, and pathology viewpoints and it was well recognized as one of the representative research results of Chinese medicine recipes for treating angiogenesis. Thus, QLY, as a traditional Chinese medicine having prominent characteristics and addressing both the symptoms and root causes for treating angiogenesis-related diseases (such as rheumatoid arthritis), has strong growing momentum and wide growing prospect.

However, the ingredients in QLY is very complex, and the pharmacological effect of QLY is not produced just by simple addition of that of single component, but that there is a complex mechanism of interactions among the effects of ingredients. So the recipe of QLY, which takes the form of decoction pieces, has the defects of complex ingredients, difficult quality control, ambiguity in its basic effective constituent, and ill-defined mechanism of its medical effect, which commonly exists for most present traditional Chinese medicines.

Generally, prior art treatment of angiogenesis-related diseases, taking rheumatoid arthritis as an example, has the current situations of:
(1) no Chinese patent medicines for treating rheumatoid arthritis characterized by anti-angiogenesis;
(2) no patented traditional Chinese medicine recipe for anti-angiogenesis-related-diseases in the form of a composition of effective constituents; and
(3) the demand far from being met for treatment of angiogenesis related diseases such as rheumatoid arthritis with an increasing patient group.

### SUMMARY OF THE INVENTION

An aim of the present invention is to overcome the above defects of the prior art by proposing a composition as effective constituent of a traditional Chinese medicine that has anti-angiogenesis synergy effect. This composition is extracted from the compound decoction pieces of QLY, has synergic actions of efficacy enhancing and toxicity reducing, has obvious therapy effect to angiogenesis-related diseases such as rheumatoid arthritis and cancer, has clear substance basis, and maintain the characteristic of traditional Chinese medicines of recipe association.

In one aspect of the present invention, there is provided a composition as effective constituent of a medicine having anti-angiogenesis synergy effect, wherein said composition is composed of:

at least one substance selected from a group consisted of matrine and oxymatrine; and

at least one substance selected from a group consisted of sinomenine and sinomenine hydrochloride.

In a further aspect of the present invention, the equivalent content proportion of matrine and sinomenine hydrochloride in said composition is:
matrine:sinomenine hydrochloride=1:0.05 to 1.

In another aspect of the present invention, there is proposed use of a composition as effective constituent of a medicine having anti-angiogenesis synergy effect, wherein said composition is composed of:

at least one substance selected from a group consisted of matrine and oxymatrine; and

at least one substance selected from a group consisted of sinomenine and sinomenine hydrochloride.

In a further aspect of the present invention, the equivalent content proportion of matrine and sinomenine hydrochloride in said composition is:

matrine:sinomenine hydrochloride=1:0.05 to 1.

In a further aspect of the present invention, the clinical one-time dosage of said composition is:

total amount of matrine is 25 mg to 383 mg, while the corresponding sinomenine hydrochloride amount is in the range of 1 mg to 383 mg.

The recipe can also be applied to other objects (animals, cells, etc.), and its dosage can be calculated by pathological empirical formula.

The recipe of composition of the present invention can be used to prepare various dosage forms of medicines through conventional method.

The composition of matrine and sinomenine hydrochloride (and/or sinomenine) of the present invention has therapy effect on angiogenesis-related diseases such as rheumatoid arthritis and cancer; moreover, a synergic action, which results in efficacy enhancing and toxicity reducing, exists between the matrine and sinomenine.

Matrine, or (+)-Matrine or α-Matrine as it is otherwise called, has a molecular formula of C15H24N2O, a molecular weight of 248.36, and the molecular structure as showed below.

Oxymatrine, or Ammothamnine as it is otherwise called, has a molecular formula of C15H24N2O2, a molecular weight of 264.36, and the molecular structure as showed below. Oxymatrine can be converted into matrine in the humane body.

Sinomenine, or Cucoline, or Kukoline as it is otherwise called, has a molecular formula of C19H23N04, a molecular weight of 329.39, and the molecular structure as showed below.

Sinomenine is commercially available in the form of its salt, such as sinomenine hydrochloride, which has the same medical effect as sinomenine. 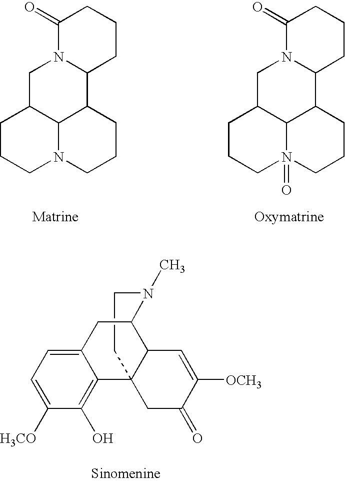
Features and Effects of the Present Invention

(1) The inventors used liquid chromatography-diode array detector-mass spectrometer (LC-DAD-MS) technique to analyze pharmaceutical ingredients in animal plasma at different time points after oral administration of QLY decoction and found that matrine and sinomenine were the main components of QLY that could be absorbed into blood through metabolic process. Meanwhile, cell experiments showed that matrine and sinomenine hydrochloride had relatively strong pharmacological activities of anti-angiogenesis, and combined use of matrine and sinomenine hydrochloride with suitable ratio had synergic effect which enhanced efficacy and reduced toxicity. Thus, the inventors determined that matrine and sinomenine were the major effective constituents of QLY.

(2) A high performance liquid chromatography (HPLC) method was developed, validated, and used to determine contents of matrine and oxymatrine in decoction of QLY. Considering that oxymatrine was converted into matrine within the living body with an estimated conversion rate range of 0%-100%, the inventors concluded that the effective dosage of matrine for oral administration of decoction of a dose of QLY containing 15 g of Sophora Root was in a range of 38 mg to 230 mg. Since Sophora Root in QLY has a clinical dosage range of 10 g to 25 g, prescribed to be taken at one time, the inventors therefore concluded that a possible one-time oral administration dosage of matrine was in the range of 25 mg to 383 mg.

(3) The inventors performed typical angiogenesis-related experiments of endothelial cells and found that both matrine and sinomenine hydrochloride had remarkable effect on suppressing proliferation of endothelial cells. The inventors also carried out cell experiments on combining use of matrine and sinomenine hydrochloride and used internationally-recognized equivalent line method to evaluate the interaction of suppression effects of matrine and sinomenine hydrochloride. The inventors found that matrine and sinomenine hydrochloride, with a proportion range of 1:(0.05˜1), at for example each of 1:0.05, 1:0.22, 1:0.44, and 1:1, showed remarkable synergy effect (please see experiment 3 below for details). These results indicated that some combinations of matrine and sinomenine hydrochloride had synergy effect which resulted in enhanced therapy efficacy and reduced toxicity; that is, a composition of matrine and sinomenine hydrochloride in proper ratio not only had improved anti-angiogenesis effect but also could effectively avoid toxicity possibly brought about by over-dosage of matrine. The inventors, through researches of animal experiments, found that compositions of matrine and sinomenine hydrochloride in proper ratios had therapy effects equivalent to QLY and in some aspects superior to control groups which took QLY and to control groups which took Tripterygium Glycosides Tablet.

### EMBODIMENTS

### Embodiment 1

Recipe of composition in a dosage: matrine at 2000 μg/ml and sinomenine hydrochloride at 100 μg/ml, so amounts of matrine and sinomenine hydrochloride had a proportion of 1:0.05. This embodiment showed the strongest synergy effect in suppressing endothelial cell proliferation, with a suppression rate of 80.16%.

### Embodiment 2

Recipe of composition in a dosage: matrine at 1350 μg/ml and sinomenine hydrochloride at 300 μg/ml, so amounts of matrine and sinomenine hydrochloride had a proportion of 1:0.22. This embodiment showed synergy effect in suppressing endothelial cell proliferation, with a suppression rate of 72.02. %.

### Embodiment 3

Recipe of composition in a dosage: matrine at 888 μg/ml and sinomenine hydrochloride at 394 μg/ml, so amounts of matrine and sinomenine hydrochloride had a proportion of 1:0.44. This embodiment showed synergy effect in suppressing endothelial cell proliferation, with a suppression rate of 70.91. %.

### Embodiment 4

Recipe of composition in a dosage: matrine at 445 μg/ml and sinomenine hydrochloride at 445 μg/ml, so amounts of matrine and sinomenine hydrochloride had a proportion of 1:1. This embodiment showed synergy effect in suppressing endothelial cell proliferation, with a suppression rate of 54.12%.

### EXPERIMENTS AND EFFECT OF THE PRESENT INVENTION

### Experiment 1

### Study on the Compounds in QLY that could be Absorbed into Rat Plasma

Description of Experiment:

Liquid chromatography simultaneously coupled with a diode array detector and a mass spectrometry detector (LC-DAD-MS) was used to analyze the pharmaceutical ingredients in rat plasma at different time points after oral administration of QLY decoction in order to determine the ingredients in QLY that could be absorbed into the body through metabolism. In this experiment, performed were comparison analyses of chromatogram and mass spectrum of QLY decoction and those of decoction of each of the constituent herbs of QLY, comparison analyses of chromatogram and mass spectrum of QLY decoction and those of QLY decoction plus blank plasma, comparison analyses of chromatogram and mass spectrum of blank plasma and those of post-administration plasma (blood sample taken at 20 min, 1 h, and 2 h respectively after oral administration of QLY decoction).

Result:

The inventors detected m/z (mass-to-charge ratio) 330 (sinomenine, [M+1]) and m/z 249 (matrine, [M+1]) as well as small amounts of m/z 205 (N-methylcytisine, [M+1]) and m/z 247 (sophocarpine, [M+1]) in plasma of rats 20 minutes after oral administration of QLY. Therefore, sinomenine and matrine were the main ingredients in QLY that could be absorbed into the animal body. From this, the inventors conducted further analysis of rat plasma taken at different time intervals after oral administration of QLY and confirmed that the major components that had gone into the rat blood through metabolism were sinomenine and matrine, thus the inventors confirmed that the major active ingredients in QLY probably included matrine and sinomenine. This provided a solid scientific basis for further study on the pharmacological effect of the combined use of the two compounds.

### Experiment 2

### Determination of Matrine and Oxymatrine Content in QLY Decoction

Description of Experiment:

The inventors, using HPLC, determined the contents of matrine and oxymatrine, which could be converted into matrine in living body, in decoction of QLY (containing 15 g of Sophora Root). The analytical standards of matrine and oxymatrine used for the assay were supplied by National Institute for the Control of Pharmaceutical and Biological Products (Beijing, China). A HPLC method was developed and validated in the present experiment with good linearity of response. The coefficient of correlation (R²) was greater than 0.9999. Both the precision and relative standard deviation (RSD) of stability test were within 3% and the recovery was over 95%.

Result:

The assay results, obtained by the inventors, of matrine and oxymatrine in decoction of QLY (containing 15 g of Sophora Root) were showed in table 1 below. The contents of matrine and oxymatrine were determined by using the validated HPLC method. The contents of matrine and oxymatrine in decoction of QLY were 38 mg and 192 mg respectively with a dilution factor of 5 and a total volume of 500 ml. Considering that oxymatrine was converted into matrine, and taking a conversion rate in the range of 0-100%, the effective content of matrine in QLY was estimated to be 38 mg to 230 mg. Since clinical dosage of Sophora Root in QLY was in a range of 10 g to 25 g (15 g of Sophora Root in QLY in this assay experiment), it was determined that clinical one-time oral administration dosage of matrine be in a range of 25 mg to 383 mg.

TABLE 1
Assay results of matrine and oxymatrine in decoction
of QLY (containing 15 g of Sophora Root)

| | linearity | | Preci- | Recov- | Concen- | Con- |
|---|---|---|---|---|---|---|
| | range | | sion | ery | tration | tent |
| Analytes | (μg/ml) | R² | (%) | (%) | (μg/ml) | (mg) |
| Oxy- | 4.59-255.0 | 0.99998 | 1.2 | 99.6 | 76.6 | 192 |
| matrine | | | | | | |
| matrine | 1.886-104.8 | 0.99999 | 2.4 | 97.3 | 15.3 | 38 |

The dilution factor is 5 and the total volume is 500 ml

### Experiment 3

### Cell Experiments on Synergy Effect of Composition of Matrine and Sinomenine Hydrochloride

Description of Experiment:

First, cell experiments were performed on separate uses of matrine and sinomenine hydrochloride, and then cell experiments on combined use of matrine and sinomenine hydrochloride in different ratios were carried out for evaluating the synergy effect of anti-angiogenesis. Human Umbilical Vein Endothelial Cells (HUVEC) were employed in the experiment and the suppression rate on endothelial cell proliferation were monitored. Classical methods including equivalent curve method and the response surface method were used and the amount-effect curves were plotted. The half maximal inhibitory concentration (IC50) of endothelial cell proliferation was also obtained to evaluate synergy effect of the composition of matrine and sinomenine hydrochloride.

Results:

The inventors found that matrine and sinomenine hydrochloride had remarkable synergy effect of anti-angiogenesis with enhanced therapy effect and reduced toxicity. Specifically:

at the proportion of matrine vs. sinomenine hydrochloride of 1:0.05 (matrine at 2000 μg/ml and sinomenine hydrochloride at 100 μg/ml), the strongest tested synergy effect on the suppression of endothelial cell proliferation was observed, with a suppression rate of 80.16%, which corresponded to the effect of administration of only matrine at 5097.7 μg/ml;

at the proportion of matrine vs. sinomenine hydrochloride of 1:0.22 (matrine at 1350 μg/ml and sinomenine hydrochloride at 300 μg/ml, synergy effect on the suppression of endothelial cell proliferation was observed, with a suppression rate of 72.02%, which corresponded to the effect of administration of only matrine at 4441.4 μg/ml;

at the proportion of matrine vs. sinomenine hydrochloride of 1:0.44 (matrine at 888 μg/ml and sinomenine hydrochloride at 394 μg/ml), synergy effect on the suppression of endothelial cell proliferation was observed, with a suppression rate of 70.91%, which corresponded to the effect of administration of only matrine at 4351.9 μg/ml; and

at the proportion of matrine vs. sinomenine hydrochloride of 1:1 (matrine at 445 μg/ml and sinomenine hydrochloride at 445 μg/ml), synergy effect on the suppression of endothelial cell proliferation was observed, with a suppression rate of 54.12%, which corresponded to the effect of administration of only matrine at 2998.2 μg/ml.

### Experiment 4

### Study on Therapy Effect of Composition of Matrine and Sinomenine Hydrochloride On Rat Collagen-Induced Arthritis (CIA)

Description of Experiment:

An internationally-recognized model of Collagen-induced arthritis (CIA) rats was used; forty rats were divided into 5 groups each of 8 rats: normal control group, model group (CIA group), Tripterygium Glycosides Tablet therapy group (where Tripterygium Glycosides Tablet was supplied by the Huangshi Factory of Sanjiu (999) Enterprise Group, Shenzhen, Guangdong Province, China), QLY therapy group, and therapy group of composition of matrine and sinomenine hydrochloride (M-S group, as called hereinbelow). Controlled experiments were performed over the 5 groups. The inventors took test indices including:

Arthritis Index (AI),

rat ankle sections, which were observed using optical microscope after Hemetoxylin and Eosin (HE) Staining for pathological changes of synovial, cartilage, and/or bone

rat serum, which was tested for anti-CII antibody concentration using ELISA method.

Results:

(1) arthritis points of CIA rats in the M-S group on the 28^{th} day, (P<0.05), 35^{th} day (P<0.01), and 42nd day were remarkably lowered, with a result superior to that of the QLY group and comparable to that of Tripterygium Glycosides Tablet therapy group.

(2) through pathology observations with optical microscope, obvious synovial hyperplasia was observed in both joint cavities and bursas in the CIA group, in which synoviocyte hyperplasia was in papillary shape, and vasodilator veins and angiogenesis as well as fibroblasts were observed in synovial tissue, and accompanied lymphocyte infiltration was observed. The matrix and chondrocyte of cartilage of each tarsal joint of ankle exhibited degeneration of various degrees, and in serious places cartilage putrescence and exfoliation were observed; on the degenerated surface, putrescent and exfoliating cartilages, pannus formed by synoviocyte and capillary vessel hyperplasia were observed; synovial tissue hyperplasia and pannus formation were observed in knee joint cavities, and degeneration of various degrees was observed on joint cartilages. In the M-S group, angiogenesis lesion of CIA and symptom of arthritis were remarkably alleviated, as were shown by decrease in number of capillary vessels, synoviocytes, and inflammatory cells in the hyperplastic synovial tissues, remarkable alleviation of hyperemia edema of synovial tissues, and remarkable suppression of angiogenesis. However, fibrous connective tissue hyperplasia still existed, and collagenization was observed. Degenerated cartilage tissues were still observed, but no new cartilage putrescence, exfoliated cartilage area, or pannus fibrosis was observed. In addition, alleviation of arthritis lesion of CIA at different levels were observed in the QLY group and the Tripterygium Glycosides Tablet group.

(3) as indicated by test results of anti-CII antibody concentration, in the M-S group, abnormally elevated anti-CII type collagen antibody concentration in the CIA rat plasma (P<0.01) were remarkably lowered, and certain improvement (P<0.01) was also observed in the QLY and Tripterygium Glycosides Tablet therapy groups respectively. All these results indicated that composition of matrine and sinomenine could effectively alleviate joint swelling of CIA rats, reduce formation of pannus, and lower anti-CII type collagen antibody concentration in CIA rat plasma, as evidented in the M-S group, so composition of matrine and sinomenine hydrochloride showed remarkable therapeutic effects on CIA rats with angiogenesis and inflammatory symptoms. Moreover, effective composition of matrine and sinomenine hydrochloride showed anti-angiogenesis therapeutic effect comparable to QLY on the CIA rats, and in some respects composition of matrine and sinomenine hydrochloride showed effect superior to QLY and Tripterygium Glycosides Tablet.

It should be noted that the present invention is not limited to the embodiments as described above. It is understood that any salt and/or compound of matrine, as long as it is converted to matrine and/or base of matrine in living body, can be taken as a component of the composition of the present invention. Similarly, it is understood that any salt and/or compound of sinomenine, as long as it is converted to sinomenine and/or base of sinomenine in living body, can be taken as a component of the composition of the present invention. Such replacement of one or more components of composition is within the scope of the present as defined by the appended claims.

What is claimed is:

1. A composition as effective constituent of a medicine having anti-angiogenesis synergy effect, said composition consisting essentially of:
at least one substance selected from a group consisting of matrine and oxymatrine; and
at least one substance selected from a group consisting of sinomenine and sinomenine hydrochloride.

2. A composition as claimed in claim 1, wherein the equivalent content ratio of matrine and sinomenine hydrochloride in said composition is:
matrine:sinomenine hydrochloride=1:0.05 to 1.

3. Use of a composition as effective constituent of a medicine having anti-angiogenesis synergy effect, said composition consisting essentially of:
at least one substance selected from a group consisting of matrine and oxymatrine; and
at least one substance selected from a group consisting of sinomenine and sinomenine hydrochloride.

4. Use as claimed in claim 3, wherein the equivalent content ratio of matrine and sinomenine hydrochloride in said composition is:
matrine:sinomenine hydrochloride=1:0.05 to 1.

5. Use as claimed in claim 3, where the clinical one-time dosage of said composition is:
total amount of matrine is 25 mg to 383 mg, while the corresponding sinomenine hydrochloride amount is in the range of 1 mg to 383 mg.
